(19) United States
(12) Patent Application Publication
(10) Pub. No.: US 2004/0082021 A1
(43) Pub. Date: Apr. 29, 2004

(54) Method for assaying compounds or agents for ability to decrease the activity of microsomal prostaglandin E synthase or hematopoietic prostaglandin D synthase

(75) Inventors: Zhuyin Li, Flemington, NJ (US); Junjie Xiong, Raritan, NJ (US); Henry Ma, Bridgewater, NJ (US); Jeffrey S. Sabol, Bridgewater, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharmaceuticals Inc., Bridgewater, NJ

(21) Appl. No.: 10/642,468

(22) Filed: Aug. 15, 2003

Related U.S. Application Data
(60) Provisional application No. 60/404,008, filed on Aug. 16, 2002.

(30) Foreign Application Priority Data
Dec. 16, 2002 (GB) 0229244.9

(51) Int. Cl. G01N 33/53; G01N 33/537; G01N 33/543

(52) U.S. Cl. 435/7.92

(57) ABSTRACT

Provided herein is a novel and useful method for evaluating the ability of compounds or agents to decrease the activity of microsomal prostaglandin E synthase or hematopoietic prostaglandin D synthase to produce their respective prostaglandin products.

### DOMESTIC PRIORITY CLAIM

This application claims priority under 35 U.S.C. § 119 of U.S. Provisional Application No. 60/404,008 filed on Aug. 16, 2002.

### PRIORITY CLAIM

This application claims priority under 35 U.S.C. § 119 of British Application No. 0229244.9 filed on Dec. 16, 2002.

### FIELD OF THE INVENTION

The present invention relates to a novel and useful method for assaying compounds or agents for their ability to decrease the activity of a prostaglandin synthase. More particularly, the present invention involves a method for assaying compounds or agents for their ability to decrease the activity of a microsomal prostaglandin E synthase (mPGES), or a hematopoietic prostaglandin D synthase (hPGDS).

### BACKGROUND OF THE INVENTION

Prostaglandins are a class of eicosanoids that play an important role in pain, fever and inflammation. They are synthesized in vivo from arachidonic acid, and possess a five-membered ring of carbon atoms that had formed part of the carbon chain of arachidonic acid. Prostaglandins are not hormones, and act locally, i.e., near the site of their synthesis.

Two prostaglandins, PGE₂ and PGD₂, play particularly important roles in the fever, pain and inflammation. In particular, upon antigenic challenge, mast cells produce increased levels of PGD₂, which is a major mediator of airway allergic disorders. In particular, it causes, among other things, bronchoconstriction, bronchial hyperactivity, nasal block, and eosinophil and TH₂ cell infiltration. PGE₂ is a pain mediator, and has been shown to play a role in inducing hyperalgesia, fever, vasodilation, and edema.

In their synthesis in vivo, phospholipase A2 converts phospholipid into arachidonic acid (found in vivo in an ester form). Subsequently, prostaglandin endoperoxide synthase converts arachidonic acid into prostaglandin G₂ (PGG₂). Prostaglandin endoperoxide synthase also catalyzes the reduction of the peroxide group on PGG₂ to form prostaglandin H₂ (PGH₂), which is the precursor of both PGE₂ and PGD₂. In the case of the production of PGE₂, prostaglandin E synthase (PGES) converts PGH₂ into PGE₂ in the presence of cofactor glutathione (GSH). There are at least two PGE₂ synthases, cytosolic PGES (cPGES) and microsomal PGES (mPGES). Both PGESs are widely expressed in many tissues with overlapping distribution. mPGES is inducible by inflammatory stimuli such as lipopolysaccharide (LPS), IL-1 and TNF-α, while cPGES is constitutively expressed. Moreover, mPGES has been shown to be coupled with COX-2 activity [Murakami et al., J. Biol. Chem. 275:32783 (2000)].

PGD₂ is produced as a result of prostaglandin D synthase's (PGDS) conversion of PGH₂ into PGD₂. Two types of PGD₂ synthases are known. The first is a lipocalin-type PGDS (L-PGDS) found mainly in the central nervous system, and the second is hematopoietic PGDS (hPGDS), which is found primarily in peripheral tissues. L-PGDS is glutathione (GSH) independent while hPGDS is GSH-dependent with GST activity. Furthermnore, there is very little structural homology between L-PGDS and hPGDS.

Since the PGD₂ and PGE₂ play an important role in fever, pain and inflammation, efforts have been made to create assays for compounds that may decrease or even inhibit their production. In particular, techniques such as HPLC, ELISA or RIA have been used to quantify the production of PGD₂ and PGE₂ in order to determine a compound's or agent's ability to decrease or inhibit the activity of a prostaglandin synthase. However, these techniques possess inherent limitations. For example, they require various washing steps, purification steps and/or usage of radioactive materials. Also, these methods are time-consuming and only have a throughput of tens (HPLC) to several hundred (ELISA & RIA) data points per day. Thus, they are not amenable to high throughput screening.

Fluorescence polarization is a technique that is used to study interactions among molecules. The principles behind this technique are dependent upon the size of molecules being evaluated. In particular, when a fluorescent molecule is illuminated with plane polarized light, electrons at their ground state in the molecule are promoted to an excited state. After approximately 4-5 nanoseconds, these excited electrons decay back to their ground state. It is during this decay that the molecule emits a fluorescence signal. In fluorescence polarization, this fluorescence emission can be detected in the same plane only if the molecule remains stationary throughout the excited state. If the molecule moves or rotates during the excited state, the fluorescence emission will be in a different plane of light than that of the polarized light that excited the electrons of the fluorescent aspect. As a result, a fluorescence emission will not be detected. It is well accepted that the smaller the molecule, the greater its mobility and rotation. Hence, a small molecule will produce a substantially smaller signal than a larger molecule, which will remain relatively stationary during the excitation period. It is this property of molecules that fluorescence polarization utilizes. In particular, in a fluorescence polarization assay of a ligand, the ligand, a tracer, i.e., the ligand labeled with a fluorescence label, and the receptor to which the ligand binds, are placed in solution. The ligand and the tracer then compete with each other to bind to the receptor. The solution is then illuminated with plane polarized light, and a signal is then detected. If there is not much ligand present in the solution, the majority of receptors present will bind to the tracer. Since the receptor is a large molecule (relative to the ligand), signal will be obtained from the fluorescence of the label a high degree of fluorescence polarization will be obtained. In contrast, if there is a large amount of ligand present, then a majority of receptors will bind with the ligand. As a result, a fluorescence polarization signal produced by the tracer alone, if produced at all, will be substantially smaller than the previously obtained signal produced by the tracer bound to the receptor. It is the difference between these signals that enable one of ordinary skill in the art to determine whether the ligand is present, and its concentration. Fluorescence polarization is measured in millipolarization units, or mP.

Thus, fluorescence polarization can be performed much more simply and efficiently than assay methods, such as ELISA, HPLC and RIA. Moreover, it readily lends itself to high throughput screening of a large number of compounds or agents in a very short period of time.

Accordingly what is needed is a fluorescence polarization method for evaluating compounds or agents for their ability to decrease or even inhibit the activity of a prostaglandin synthase, and particularly for evaluating whether an agent or compound that decreases or inhibits the ability of mPGES to produce PGE₂, and for evaluating whether an agent or compound that decreases or inhibits the ability of hPGDS to produce PGD₂.

What is also needed is a high throughput system for evaluating the ability of compounds or agents to decrease or inhibit the activity of a prostaglandin synthase, and particularly an mPGES or an hPGDS. Compounds that decrease or even inhibit the activity of hematopoietic prostaglandin D₂ synthase (HPGDS) or inducible microsomal prostaglandin E₂ synthase (mPGES) may have applications in treating inflammation and allergy symptoms, such as arthritis, asthma, and rhinitis, to name only few. Moreover, pain and/or fever may also be treated with such a compound or agent.

The citation of any reference herein should not be construed as an admission that such reference is available as “Prior Art” to the instant application.

### SUMMARY OF THE INVENTION

There is provided, in accordance with a present invention, a new and useful method of evaluating compounds or agents for their ability to decrease or even inhibit the activity of MPGES or HPGDS to produce their respective prostaglandin products, which does not utilize radioactive isotopes, does not require numerous washing steps, and can be performed in vitro, ex vivo, in a cell based manner, or in an isolated manner. Moreover, a method of the present invention can readily be performed in a high throughput manner.

Broadly, the present invention extends to a method for determining whether a compound or agent decreases the activity of a prostaglandin synthase to react with its substrate to form a prostaglandin product, wherein the prostaglandin synthase is selected from the group consisting of microsomal prostaglandin E synthase (mPGES) and hematopoietic prostaglandin D synthase (hPGDS). Such a method of the present invention comprises the steps of mixing the prostaglandin synthase with its substrate, a cofactor and the compound or agent so that the enzymatic reaction can occur. The mixture is then incubated with a stop solution comprising an agent that prevents the spontaneous conversion of unreacted substrate into the prostaglandin product. This mixture is then incubated with a detection reagent that comprises the prostaglandin product labeled with a fluorescence label (i.e. a tracer), and an antibody having the prostaglandin product as an immunogen. Subsequently, the mixture and a control mixture that has been treated in the identical fashion, but lacks the compound or agent, are illuminated with plane polarized light having a wavelength at which the fluorescence label fluoresces. The fluorescence polarization of the mixture and the control mixture are measured and compared. A mixture having a polarization measurement greater than the polarization measurement of the control mixture indicates that the compound or agent decreased the activity of the prostaglandin synthase. Consequently, such a compound or agent may readily have applications in treating a subject suffering from inflammation, allergy, pain and fever, or any combination thereof, to name only a few.

Furthermore, the present invention extends to a method such as described above, wherein the prostaglandin synthase is inducible microsomal prostaglandin E synthase (mPGES), the substrate is prostaglandin H₂ (PGH₂), the cofactor is glutathione (GSH), and the prostaglandin product is prostaglandin E₂ (PGE₂). The mPGES used in a method of the present invention can be bovine, ovine, rodent, equine, canine, human, feline, etc. In a particular embodiment, the MPGES is human, and has the amino acid sequence set forth in FIG. 9 B and SEQ ID NO:2, In addition, the mPGES used in a method of the present invention need not be in a purified form.

The present invention further extends to a method for determining whether a compound or agent decreases the activity of a prostaglandin synthase to react with its substrate to form a prostaglandin product as described herein, wherein the prostaglandin synthase is hematopoietic prostaglandin D synthase (hPGDS), the substrate is PGH₂, the cofactor is GSH, and the prostaglandin product is prostaglandin D₂ (PGD₂). Just as with mPGES, the hPGDS having applications in a method of the present invention can be obtained from numerous sources. In a particular embodiment, the hPGDS is human hPGDS and has the amino acid sequence set forth in FIG. 10B, and SEQ ID NO:4. Moreover, the hPGDS need not be in a purified form.

As explained above, in a method of the present invention, the stop solution comprises an agent that prevents the spontaneous conversion of unreacted substrate into prostaglandin product. In particular, the substrate for the two prostaglandin synthases described above, PGH₂, contains a peroxide group. Although under no obligation to explain their mechanisms and certainly not intending to be bound by any explanation, it is believe that both mPGES and hPGDS catalyze the rupture of the oxygen bond of the peroxide group of PGH₂, and convert PGH₂ into PGE₂ and PGD₂, respectively. However, PGH₂ also undergoes spontaneous conversion into PGE₂ or PGD₂. This spontaneous conversion can interfere with and alter the results of an assay for a compound's or agent's ability to decrease the activity of mPGES or hPGDS. Thus in a method of the present invention, the mixture is incubated with a stop solution comprising an agent that prevents the spontaneous conversion of PGH₂ into either PGD₂ or PGE₂. A particular example of such an agent is FeCl₂ at a concentration of about 20 mM. However, one of ordinary skill in the art may readily be familiar with other agents that prevent this simultaneous conversion, and which are encompassed by a method of the present invention. Moreover, the duration of this incubation can vary, but must be sufficiently long to prevent the conversion of any remaining unreacted PGH₂ into prostaglandin product.

Furthermore, the present invention extends to a method for determining whether a compound or agent decreases the activity of a prostaglandin synthase, and particularly mPGES or hPGDS, to react with its substrate to form a prostaglandin product, wherein after incubation with the stop solution, the mixture is incubated with a detection reagent that comprises the prostaglandin product labeled with a fluorescence label, and an antibody having the having the prostaglandin product as an immunogen. Numerous fluorescence labels known to those of ordinary skill in the art have applications in a method of the present invention. Examples of such fluorescence labels include fluorescein, phycoerythrin (PE), Texas red (TR), rhodamine, a free lanthanide series salt, a chelated lanthanide series salt, CyDye (Amersham Biotech), BODIPY (Molecular Probes) and ALEXA (Molecular Probes), to name only a few. In a particular embodiment, the fluorescence label is Texas red. Moreover, the fluorescence label may be bound directly to the prostaglandin product, or alternatively bound to a linker molecule, which in turn is bound to the prostaglandin product. Particular linker molecules having applications herein include, but certainly are not limited to aminobutyric acid, aminocaproic acid, 7-aminoheptanoic acid, 8-aminocaprylic acid, Fmoc-aminocaproic acid, one or more β-alanines, an isothiocyanate group, a succinimidyl ester, a sulfonal halide or a carboduimide, to name only a few. In a particular embodiment of the present invention, the prostaglandin product is bound to a carboduimide linker, which in turn is bound to the fluorescence label, e.g. Texas red.

In addition, the present invention extends to a method for determining whether a compound or agent decreases or inhibits the reaction of inducible microsomal prostaglandin E synthase (mPGES) with its prostaglandin H2 (PGH₂) substrate to form prostaglandin E₂ (PGE₂), comprising the steps of:

(a) mixing mPGES with PGH₂, glutathione (GSH) and the compound or agent for at least 30 seconds;

(b) incubating the mixture of step (a) with a stop solution comprising FeCl₂;

(c) incubating the mixture of step (b) with a detection reagent comprising PGE2 labeled with Texas Red, and an antibody having PGE₂ as an immunogen;

(d) illuminating the mixture of step (c) and a control mixture with plane polarized light at a wavelength of 580±20 nm and measuring the fluorescence polarization of the mixture of step (c) and the control mixture; and

(e) comparing the measurements of step (d).

If the fluorescence polarization measurement of the mixture containing the compound or agent being assayed is greater than the fluorescence polarization measurement of control mixture, it is indicative that the compound or agent decreases the activity of MPGES. In a particular embodiment, the mPGES is human mPGES having the amino acid sequence set forth in FIG. 9B and SEQ ID NO:2, and a carboduimide linker is bound to the PGE₂ and the Texas Red. The wavelength at which Texas red is excited is 580 nm. However, this wavelength can range up to ±20 nm from 580 nm. Similarly, the wavelength of the fluorescence emission of Texas Red is generally believed to be 620 nm. However, this wavelength can vary up to ±20 nm 620 nm. Such variations in the excitation wavelength and emission wavelength are encompassed by the present invention. Moreover, as explained previously, the mPGES need not be in a purified form.

In addition, the present invention extends to a method for determining whether a compound or agent decreases the reaction of hematopoietic prostaglandin D synthase (HPGDS) with its prostaglandin H₂ (PGH₂) substrate to form prostaglandin D₂ (PGD₂), comprising the steps of:

(a) mixing hPGDS with PGH₂, GSH and the compound or agent for at least 30 seconds;

(b) incubating the mixture of step (a) with a stop solution comprising FeCl₂;

(c) incubating the mixture of step (b) with a detection reagent comprising PGD₂ labeled with Texas Red, and an antibody having PGD₂ as an immunogen;

(d) illuminating the mixture of step (c) and a control mixture with linearly polarized light at a wavelength of 580±20 nm and measuring the fluorescence polarization of the mixture of step (c) and the control mixture; and

(e) comparing the measurements of step (d).

If the fluorescence polarization measurement of the mixture containing the compound or agent being assayed is greater than the fluorescence polarization measurement of the control mixture, it is indicative that the compound or agent decreases the activity of hPGDS. In a particular embodiment, the hPGDS is human hPGDS having the amino acid sequence set forth in FIG. 10B and SEQ ID NO:4, and the Texas Red and the PGD₂ are chemically linked via a carbodiimide linker.

Furthermore, the present invention extends to a method for determining whether a compound or agent decreases the activity of a prostaglandin synthase, e.g. mPGES or hPGDS, to produce a prostaglandin product, wherein such a method is performed in a high throughput manner.

Accordingly, it is an aspect to provide a method for evaluating the ability of compounds or agents to decrease or even inhibit the activity of a prostaglandin synthase, e.g. mPGES or hPGDS. Consequently, a method of the present invention permits one of ordinary skill in the art to identify a compound or agent that may have applications in treating pain, inflammation, fever, or a combination thereof in a subject.

It is another aspect of the present invention to provide a method for evaluating the ability of compounds or agents to decrease or inhibit the activity of mPGES or hPGDS, wherein such a method does not require washing steps or the use of radioactive isotopes.

It is still another aspect of the present invention to provide a method for evaluating the ability of compounds or agents to decrease or inhibit the activity of a prostaglandin synthase, such as mPGES or hPGDS, that can be performed ex vivo, in vitro, cell based, or in an isolated fashion.

It is yet still another aspect of the present invention to provide a method for evaluating the ability of compounds or agents to decrease or inhibit the activity of a prostaglandin synthase that can be performed in a high throughput manner.

These and other aspects of the present invention will be better appreciated by reference to the following drawings and Detailed Description.

### BRIEF DESCRIPTION OF THE DRAWINGS

FIG. 1 is a schematical view of competition between enzymatic conversion of substrate PGH₂ to PGE₂ and the spontaneous conversion of PGH₂ into either prostaglandin product PGD₂ or PGE₂, where the enzyme utilized is mPGES, and the prevention of the spontaneous conversion is with FeCl₂.

FIG. 2 is a schematical view of a method of the present invention, wherein the prostaglandin synthase is mPGES, and the prostaglandin product is PGE₂.

FIG. 3 is a schematical view of competition between enzymatic conversion of substrate PGH₂ to PGD2 and the spontaneous conversion of PGH₂ into either prostaglandin product PGD₂ or PGE₂, where the enzyme utilized is hPGDS, and the prevention of the spontaneous conversion is with FeCl₂.

FIG. 4 is a schematical view of a method of the present invention, wherein the prostaglandin synthase is hPGDS, and the prostaglandin product is PGD₂.

FIG. 5 shows the chemical structure of MK-886, a known inhibitor of mPGES that was used to prove a method of the present invention enables one to determine whether a compound or agent decreases or inhibits the activity a prostaglandin synthase.

FIG. 6 is a graphical view of the concentration response curve of a method of the present invention using the prostaglandin synthase mPGES and a known inhibitor MK-886. IC50=27.5 uM. These results show that a method of the present invention permits one of ordinary skill in the art to determine whether a compound or agent decreases the activity a prostaglandin synthase, in this case mPGES.

FIG. 7 shows the chemical structure of HQL 79.

FIG. 8 shows a histogram showing that the decrease in activity of hPGDS caused by HQL 79 can be detected by a method of the present invention.

FIGS. 9A and 9B show the nucleotide sequence and the amino acid sequence for human MPGES used in Example I infra (SEQ ID NOS: 1 and 2, respectively).

FIGS. 10A and 10B show the nucleotide sequence and the amino acid sequence for human hPGDS (SEQ ID NOS:3 and 4, respectively).

### DETAILED DESCRIPTION OF THE INVENTION

The present invention is based upon the discovery that surprisingly and unexpectedly, fluorescence polarization can be used to identify compounds or agents that decrease the activity of a prostaglandin synthase, e.g., mPGES or hPGDS, to produce a prostaglandin, e.g., PGD₂ or PGE₂. Hence broadly, the present invention extends to a method for determining whether a compound or agent decreases the activity of a prostaglandin synthase selected from the group consisting of mPGES and hPGDS, to react with its substrate to form a prostaglandin product, comprising the steps of:

(a) mixing the prostaglandin synthase with its substrate, a cofactor and the compound or agent;

(b) incubating the mixture of step (a) with a stop solution comprising an agent that prevents the spontaneous conversion of the substrate into the prostaglandin product;

(c) incubating the mixture of step (b) with a detection reagent comprising the prostaglandin product labeled with a fluorescence label and an antibody having the prostaglandin product as an immunogen;

(d) illuminating the mixture of step (c) and a control mixture with linearly polarized light at a wavelength at which the fluorescence label fluoresces, and measuring the fluorescence polarization of the mixture of step (c) and the control mixture; and

(e) comparing the measurements of step (d), wherein the fluorescence polarization measurement of the mixture of step (d) that is greater than the fluorescence polarization measurement of control mixture indicates the compound or agent decreases the activity of the prostaglandin synthase.

Numerous terms and phrases used throughout the instant Specification and appended Claims are defined below. Accordingly:

As used herein, the terms “compound” or “agent” refer to any composition presently known or subsequently discovered. Examples of compounds or agents having applications herein include organic compounds (e.g., man made or naturally occurring), peptides (man made or naturally occurring), carbohydrates, nucleic acid molecules, etc.

As used herein, the term “enzyme” refers to a biomolecule, such as a protein or RNA that catalyzes a specific chemical reaction. It does not affect the equilibrium of the catalyzed reaction. Rather, the enzyme enhances the rate of reaction by lowering the energy of activation.

As used herein, the term “prostaglandin synthase” refers to enzymes that catalyze the conversion of prostaglandin H2 (PGH₂) into a prostaglandin product. Particular examples of prostaglandin synthases having applications in the present invention include inducible microsomal prostaglandin E synthase (mPGES), which converts prostaglandin H₂ into prostaglandin E₂ (PGE₂) in the presence of cofactor glutathione (GSH). Another example is hematopoietic prostaglandin D synthase (hPGDS), which converts prostaglandin H-2 into prostaglandin D₂ (PGD₂) in the presence of cofactor GSH.

As used herein, the term “substrate” refers to the compound an enzyme acts upon to produce the product. An example of a substrate having applications herein is PGH₂.

As used herein, the term “cofactor” refers to an organic molecule, an inorganic molecule, a peptide, or a protein required for enzyme activity. In particular embodiments of the present invention, wherein the prostaglandin synthase is mPGES or hPGDS, the cofactor is glutathione (GSH).

As used herein, the term “prostaglandin product” refers to a product produced due to action of a prostaglandin synthase on the substrate of the prostaglandin synthase. Thus, for mPGES, the prostaglandin product is PGE₂, and for hPGDS, the prostaglandin product is PGD₂.

As used herein, the term “fluorescence label” refers to substance that fluoresces when illuminated with a particular wavelength of light, that is bound directly to a compound of interest, or alternatively, to a linker that is in turn bound to the compound of interest. Examples of fluorescence labels having applications in a method of the present invention include, but certainly are not limited to fluorescein, phycoerythrin (PE), Texas red (TR), rhodamine, a free lanthanide series salt, a chelated lanthanide series salt, BODIPY, ALEXA, CyDye, etc. A particular fluorescence label having applications in a method of the present invention is Texas red.

As used herein, the terms “linker” and “linker molecule” may be used interchangeably, and refer to a chemical moiety to which the fluorescence label and the prostaglandin product are bound. Particular examples of linkers having applications in the present invention include aminobutyric acid, aminocaproic acid, 7-aminoheptanoic acid, 8-aminocaprylic acid, Fmoc-aminocaproic, one or more β-alanines, an isothiocyanate group, a succinimidyl ester, a sulfonal halide, or a carboduimide, to name only a few. A particular example of a linker having applications in the present invention is a carbodiimide group.

As used herein, the term “control mixture” refers to a mixture containing the same reagents, compounds, cells, etc. in the same amounts as the mixture containing the compound or agent being assayed, and is treated in the same manner as the mixture containing the compound or agent being assayed, except, the control mixture does not contain the compound or agent to be assayed.

Antibodies

As explained above, a method of the present invention utilizes an antibody having a prostaglandin product, such as PGD₂ or PGE₂, as an immunogen. Such an antibody can be a monoclonal antibody, a polyclonal antibody, or even a chimeric antibody. Various procedures known in the art may be used for the production of polyclonal antibodies to PGE₂ or PGD₂. For the production of antibody, various host animals can be immunized by injection with the prostaglandin product, including but not limited to rabbits, mice, rats, sheep, goats, etc. In a particular embodiment, the PGE₂ or PGD₂ can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, BCG (bacille Calmette-Guerin) or Corynebacterium parvum.

For preparation of monoclonal antibodies directed toward a prostaglandin product, any technique that provides for the production of antibody molecules by continuous cell lines in culture may be used. These include, but are not limited to, the hybridoma technique originally developed by Kohler and Milstein [Nature 256:495497 (1975)], as well as the trioma technique, the human B-cell hybridoma technique [Kozbor et al., Immunology Today 4:72 1983); Cote et al., Proc. Natl. Acad. Sci. U.S.A. 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. Furthermore, monoclonal antibodies can be produced in germ-free animals utilizing technology described in PCT/US90/02545. Techniques developed for the production of “chimeric antibodies” [Morrison et al., J. Bacteriol. 159:870 (1984); Neuberger et al., Nature 312:604-608 (1984); Takeda et al., Nature 314:452-454 (1985)] by splicing the genes from a mouse antibody molecule specific for a prostaglandin product together with genes from a human antibody molecule of appropriate biological activity can be used.

Conditions

As explained above, a method of the present invention can be performed ex vivo, in vitro, or in an isolated form, wherein all the reagents, enzymes, substrates, etc. were previously isolated and maintained in a buffer solution, such as TRIS, TRIS HCl, HEPEs, or phosphate buffer, or in a cell-based manner. Moreover, in a method of the present invention, the prostaglandin synthase need not be purified.

In a cell based assay, a method of the present invention is used to determine whether the compound or agent being assayed prevents or decreases the cell's secretion of prostaglandin product, while in an in vitro method, cells may be lysed prior to performance of a method of the present invention so that a compound or agent can be evaluated in an intracellular medium.

Search of Libraries for Candidate Compounds or Agents that Decrease or Inhibit the Activity of a Prostaglandin Synthase

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In a particular embodiment, high throughput screening methods involve providing a collection containing a large number of potential therapeutic compounds (candidate compounds). Such “historic chemical collections” are then screened with a method of the present invention to identify those members of such a collection (particular chemical species or subclasses) that display the desired characteristic activity. The compounds thus identified can serve as conventional “lead compounds” or can themselves be used as potential or actual therapeutics.

Combinatorial Chemical Libraries

Combinatorial chemical libraries are a preferred means to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. Applications of Combinatorial Technologies to Drug Discovery 2. Combinatorial Organic Synthesis, Library Screening Strategies and Future Directions J. Med. Chem. (1994) 37(9):1233-1251).

Preparation of combinatorial chemical libraries is well known to those of ordinary skill in the art. Such combinatorial chemical libraries include, but are not limited to, peptide libraries (see, e.g., U.S. Pat. No. 5,010,175, Furka (1991) Int. J. Pept. Prot. Res., 37:487493, Houghton et al. (1991) Nature, 354:84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random biooligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90:69096913), vinylogous polypeptides (Hagihara et al. (1992) J. Amer. Chem. Soc. 114:6568), nonpeptidal peptidomimetics with a Beta D Glucose scaffolding (Hirschmann et al., (1992) J. Amer. Chem. Soc. 114:92179218), analogous organic syntheses of small compound libraries (Chen et al. (1994) J. Amer. Chem. Soc. 116:2661), oligocarbamates (Cho, et al., (1993) Science 261:1303), and/or peptidyl phosphonates (Campbell et al., (1994) J. Org. Chem. 59:658). See, generally, Gordon et al., (1994) J. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3):309-314), and PCT/US96/10287), carbohydrate libraries (see, e.g., Liang et al. (1996) Science, 274:1520-1522, and U.S. Pat. No. 5,593,853), and small organic molecule libraries (see, e.g., benzodiazepines, Baum (1993) C&EN, Jan 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. Nos. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic armns (Zymate II, Zymark Corporation, Hopkinton, Mass.; Orca, HewlettPackard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

High Throughput Assays of Chemical Libraries

Naturally, a method of the present invention, which employs fluorescence polarization, is readily amenable to high throughput screening. High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for the various high throughput. Thus, for example, Zymark Corp. provides technical bulletins describing screening systems for detecting the modulation of gene transcription, ligand binding, and the like.

The present invention may be better understood by reference to the following non-limiting Examples, which are provided as exemplary of the invention. The following Examples are presented in order to more fully illustrate the preferred embodiments of the invention. They should in no way be construed, however, as limiting the broad scope of the invention.

### EXAMPLE I

Fluorescence Polarization Assay of inducible Microsomal Prostaglandin E Synthase (mPGES)

Prostaglandin E₂ (PGE₂) is a major mediator involved in inflammation and pain. Microsomal prostaglandin E synthase (mPGES) catalyzes the conversion of PGH₂ to PGE₂ in the presence of glutathione. Expression of mPGES is induced in many inflammatory conditions. Thus, a compound that decreases or inhibits the activity of MPGES will potentially be an effective therapeutic for inflammation, pain, fever, or a combination thereof, to name only a few diseases or disorders.

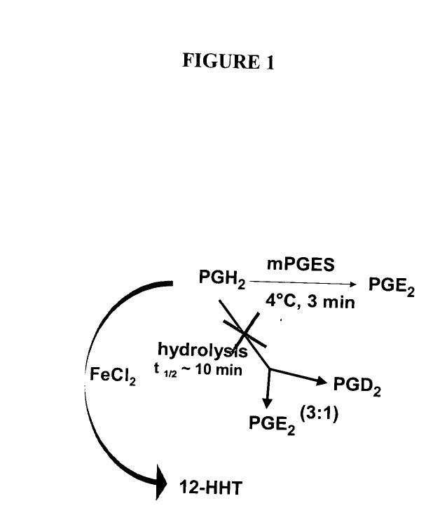

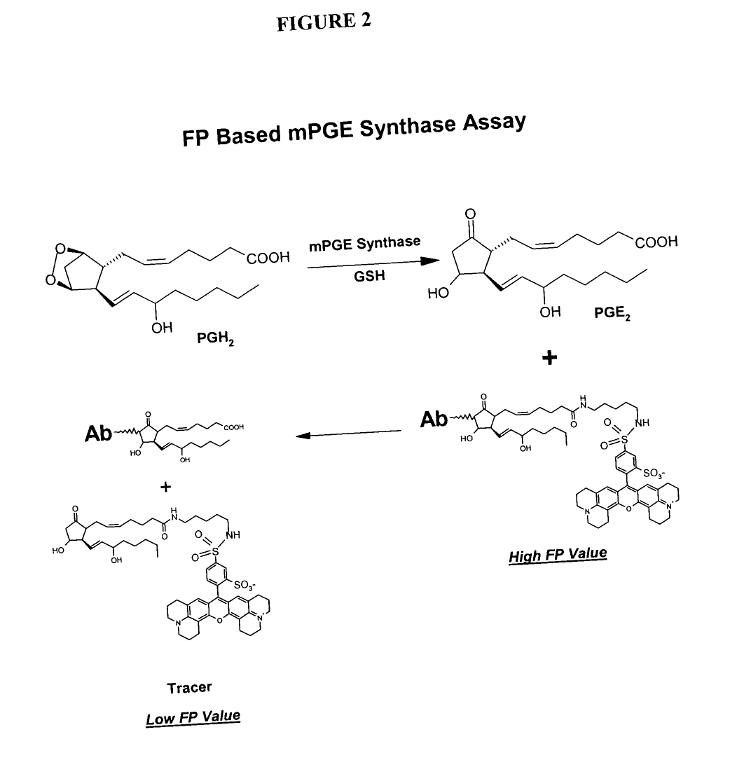

An assay has been developed to measure the conversion of PGH, to PGE₂ by inducible microsomal PGE₂ synthase. The assay is configured based on the Fluorescence Polarization principle. The enzyme is incubated with PGH₂, glutathione, and the compound or agent being evaluated. After a short incubation period (at least 30 seconds), a stop solution containing FeCl₂ and citric acid is added to quench any remaining PGH₂, which would otherwise undergo spontaneous conversion to PGD₂ or PGE₂, and thus interfere with the quantification of the enzymatic conversion of PGH₂ to PGE₂ (FIG. 1). A detection solution containing a fluorescence labeled (Texas Red) tracer (PGE₂) and anti-PGE₂ antibody is then added in order to generate the specific signal that is inversely proportional to the production of PGE₂ (FIG. 2). The PGE₂ generated from the enzymatic reaction will compete specifically for the antibody and release the fluorescence labeled tracer. Inhibition of PGE₂ synthase activity will result in increased FP value.

Materials

Glutathione (GSH):

available from Sigma (Catalog # G-6529).

PGH₂

available from Cayman Chemicals, Inc. (Catalog #17020).

PGE₂ Monoclonal Antibody

avialable from Assay Designs, Inc. (Catalog # 915-057)

PGE₂:

available from Cayman Chemicals, Inc. (Catalog #14010)

Expression of Human mPGES Enzyme

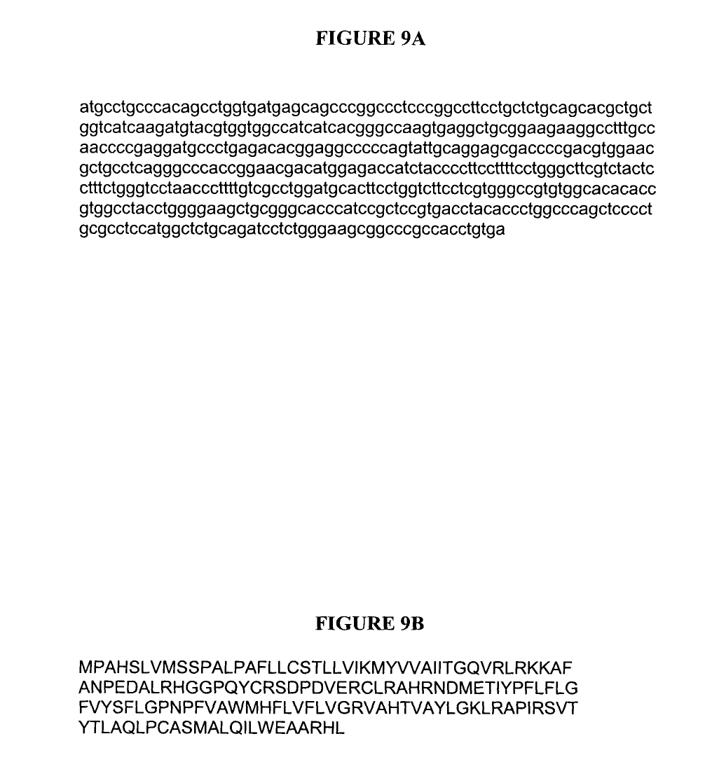

Human MPGES enzyme was expressed using a bacterial expression system and the procedures set forth in [Jakobsson, P., et al. (Identification of human prostaglandin E synthase:a microsomal glutathione-dependent, Inducible Enzyme, Constituting a Potential Novel Drug Target. Proc. Natl. Acad. Sci. USA 96:7220-7225 (June, 1999)]. Thus, the mPGES used in the instant Example was not in a purified form, but rather was contained in a membrane fraction obtained from the bacteria used to express the mnPGES. The DNA sequence that encodes the human mPGES used in the instant Example is set forth in FIG. 9A and in SEQ ID NO: 1. The amino acid sequence of the human mPGES used in the instant example is set forth in FIG. 9B and SEQ ID NO:2.

Fluorescence Labeled Prostaglandin Product PGE₂

As explained above, numerous fluorescence labels have applications in a method of the present invention. In a particular embodiment, the fluorescence label Texas Red is linked via a linker to prostaglandin E₂ (PGE₂). In this embodiment, the PGE₂ labeled with Texas Red was synthesized by Combining (San Mateo, Calif.).The mechanism for producing such a moiety is described below:

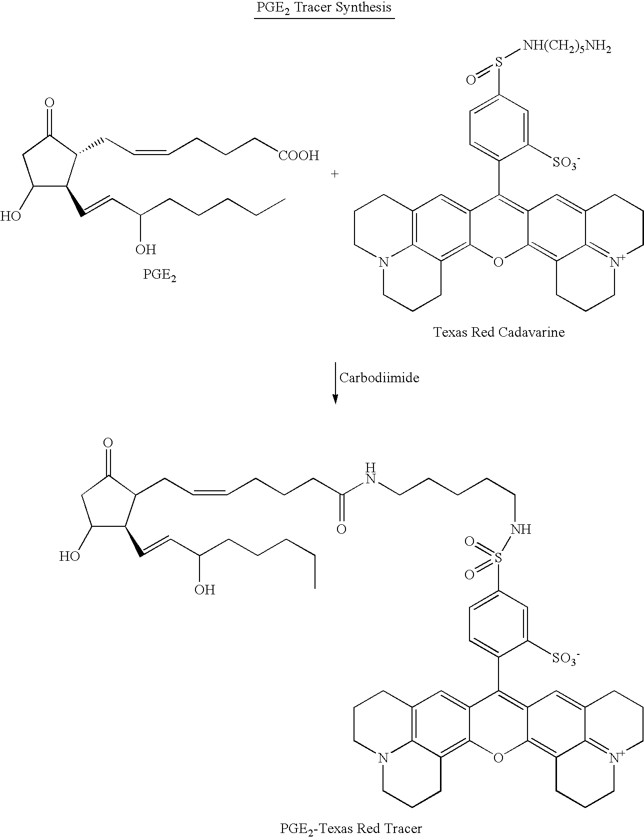

In this synthesis, Texas Red Cadavarine (Molecular Probes) was added to a solution of the prostaglandin E₂ (Cayman Chemicals) in dry methylene chloride. Dicyclohexylcarbodiimide (Sigma-Aldrich) was added and the reaction was stirred under nitrogen in the dark for 24 hours. Purification was performed by reverse phase HPLC chromatography using a water/acetonitrile gradient with 0.05% TFA as a modifier. The linker used in this synthesis can be varied.

Method

Initially, the membrane fraction containing the enzyme was diluted in reaction buffer containing K₂HPO₄ and KH₂PO₄ to make a phosphate buffered enzyme solution. The compound or agent being assayed was then placed in the enzyme solution. Optionally, this solution can then be incubated. In this particular example, the solution was incubated for about 30 minutes. The substrate PGH₂ in acetone and cofactor GSH were then placed in a separate container at 4° C. The enzyme solution was then added to the container containing PGH₂ in order to start the reaction. This mixture was incubated for about 30 seconds. Stop solution containing FeCl₂ at 20 mM was then mixed into the mixture to prevent spontaneous conversion of any remaining PGH₂ into PGE₂. Detection Solution comprising the anti-PGE₂ antibody and the PGE₂-Texas Red tracer was then added to the mixture, and the entire mixture was incubated. In this particular example, the duration of this incubation was about 120 minutes. However, a skilled artisan can vary the duration of any incubation period described in this Example and still obtain useful results. A control mixture identical to this mixture, except that the control mixture lacked the compound or agent, was treated identically, i.e., contained the same reagents, had been incubated for the same periods of time, etc. The entire mixture and the control mixture were then illuminated with plane polarized light at a wavelength of 580 nm and the fluorescence polarization of the mixture and the control mixture were measured using a fluorescence filter set with an excitation wavelength of 580 nm, and an emission wavelength of 620 nm. The measurements were made while the measuring instrument was in FP mode. These two measurements were then compared to determine whether the fluorescence polarization measurement of the mixture containing the compound or agent is greater than the fluorescence measurement of the control solution. A measurement of the mixture that is greater than a measurement of the control mixture indicates the compound or agent decreases the activity of mPGES.

Results

The assay described above was validated with a known inhibitor of MPGES. This inhibitor, MK-886 is commercially available from Biomol Research Laboratories, Inc. (Plymouth Meeting, Pa., catalog #EI-266), and has been assigned CAS# 118414-82-7.

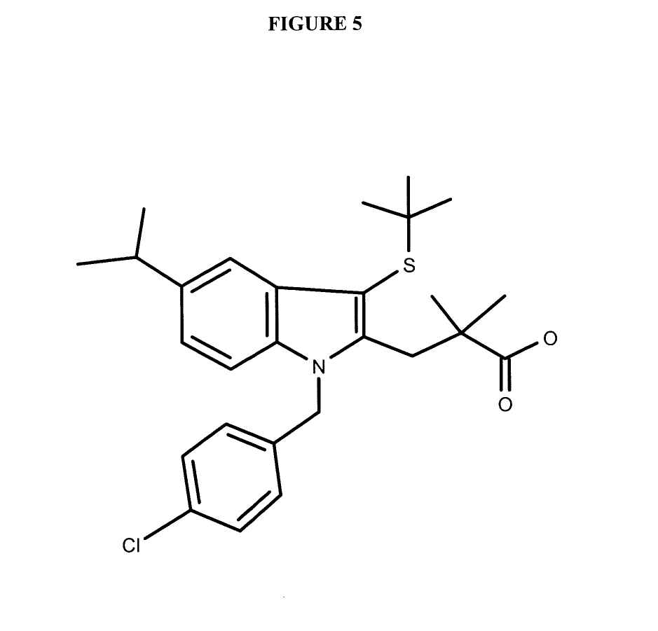

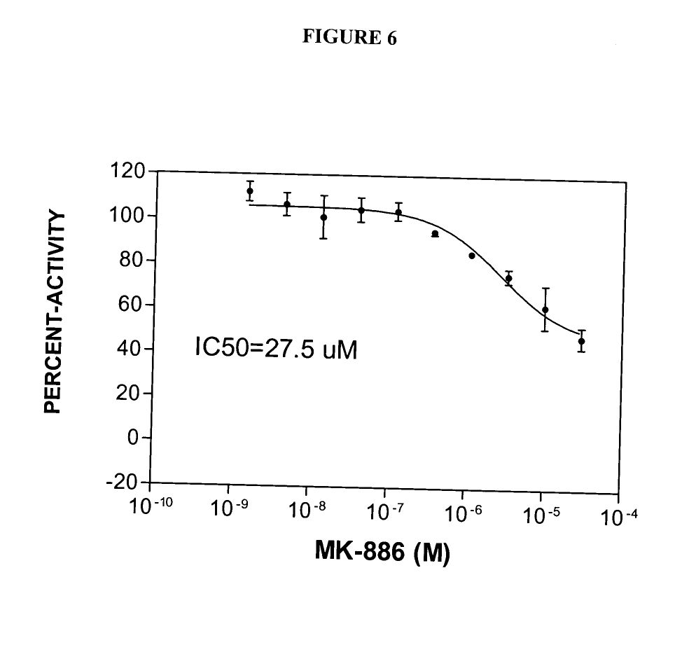

Its structure is set forth in FIG. 5. The results of this experiment are set forth in FIG. 6. These results show the concentration response curve of the MPGES assay, and clearly indicate that this method has applications in assaying a compound's or agent's ability to decrease, or even inhibit, the activity of mPGES.

### EXAMPLE II

Fluorescence Polarization Assay of Hematopoietic Prostaglandin D Synthase (hPGDS)

Antigenic challenge will increase the production of PGD₂ in airway allergic disorders. PGD₂, which is produced due to hematopoietic prostaglandin D synthase's (hPGDS) conversion of PGH₂ into PGD₂, binds to both D type prostaglandin receptor (DP) and the chemokine receptor for Th2 cells (CRTH2), and increases bronchoconstriction, vasodilation, and nasal mucosal dilation. The resulting bronchial hyperactivity, nasal blockage, and eosinophil and Th2 cell infiltration lead to allergic responses. Consequently, compounds or agents that decrease or inhibit the activity of HPGDS may readily have applications as therapeutics.

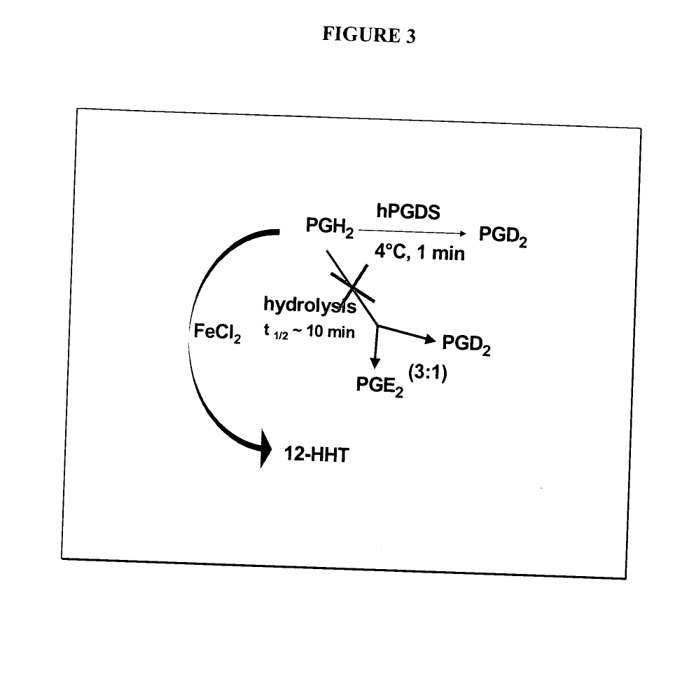

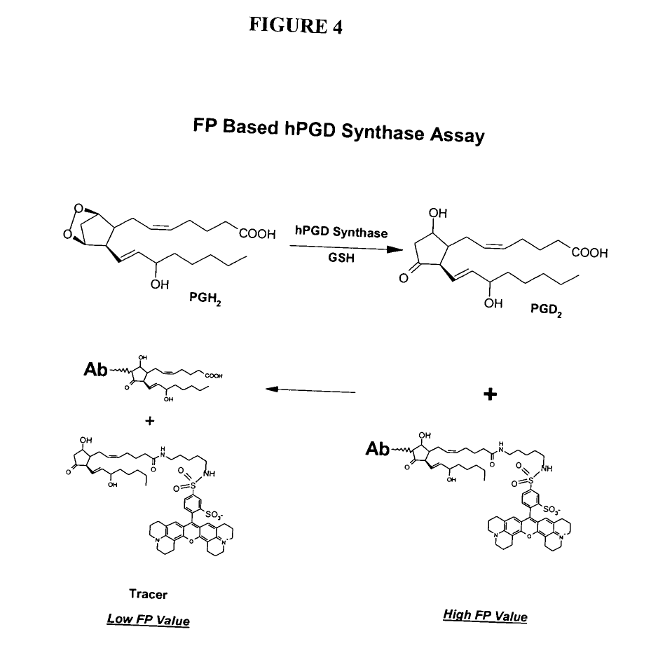

A fluorescence polarization (FP) assay to measure hPGDS activity has also been developed (FIGS. 3 and 4). The assay is configured based on the fluorescence polarization principle. hPGDS was mixed with PGH₂, glutathione, and the compound or agent being evaluated. After a short period of time (about 30 seconds), a stop solution containing FeCl₂ (20 mM) was added to quench any remaining PGH₂, which would undergo spontaneous conversion to a mixture of PGD₂ and PGE₂, and thus interfere with the quantification of the enzymatic conversion of PGH₂ to PGD₂ (FIG. 3). A detection solution containing a fluorescence labeled (Texas Red) tracer (PGD₂) and anti-PGD₂ antibody was then added in order to generate the specific signal that is inversely proportional to the production of PGD₂ (FIG. 4). The PGD₂ generated from the enzymatic reaction competed specifically for the antibody and released the fluorescence labeled tracer. For reasons discussed above, a decrease or inhibition of PGD₂ synthase activity results in increased fluorescence polarization (FP) value.

Materials

Expression of hPGDS

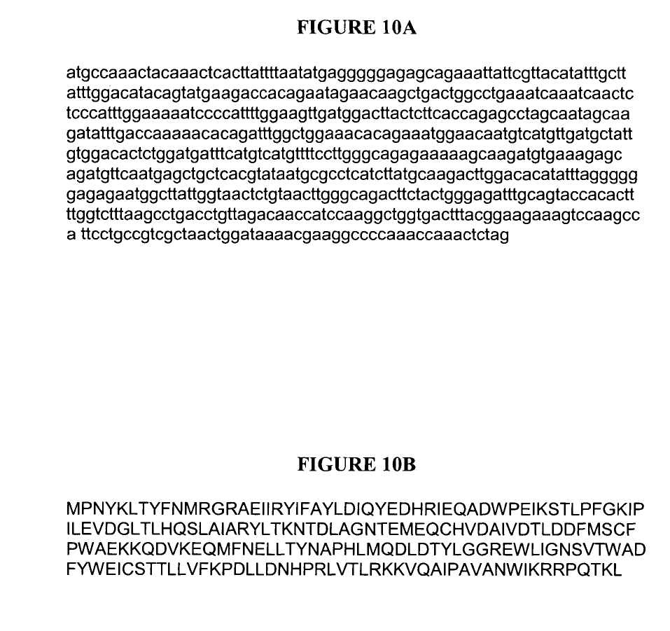

Human hematopoietic PGD₂ synthase was expressed using a bacterial expression system pursuant to the procedure set forth in Kanaoka et al. (Structure and Chromosomal Localization of Human and Mouse Genes for Hematopoietic Prostaglandin D Synthase. Eur. J. Biochem. 267:3315-3322 (2000)). The nucleotide sequence that encodes the enzyme is set forth in FIG. 10A and SEQ ID NO:3. The coding nucleotide sequence was amplified and then inserted into a pT7-7 vector, which in turn was used to transform cells of the E. coli FL21 (DE3) strain. Thio-β-D-galactoside at a final concentration of 0.6 mM was then added to the transformed cells in order to induce production human hPGDS enzyme. The human hPGDS was purified by GSH-Sepharose 4B column chromatography The amino acid sequence of the human hPGDS used in the instant example is set forth in FIG. 10B and SEQ ID NO:4.

GSH

available from Sigma (Catalog # G-6529)

PGH₂

Purchased from Cayman Chemical Inc. (Ann Arbor, Mich.) and assigned catalog No. 17020.

Anti-PGD₂ Antibody

Monoclonal anti-PGD₂ antibody was purchased from Institute Pasteur and assigned catalog number 0465328.

PGD₂:

Was available from Cayman Chemicals, Inc. (Ann Arbor, Mich.) (Catalog #12010).

Fluorescence Labeled Prostaglandin Product PGD₂

Just as in Example 1 above, Texas Red was used as the fluorescence label. The prostaglandin D₂ (PGD₂) labeled with Texas red was also produced by Combinix (San Mateo, Calif.). The mechanism for producing such a moiety is described below:

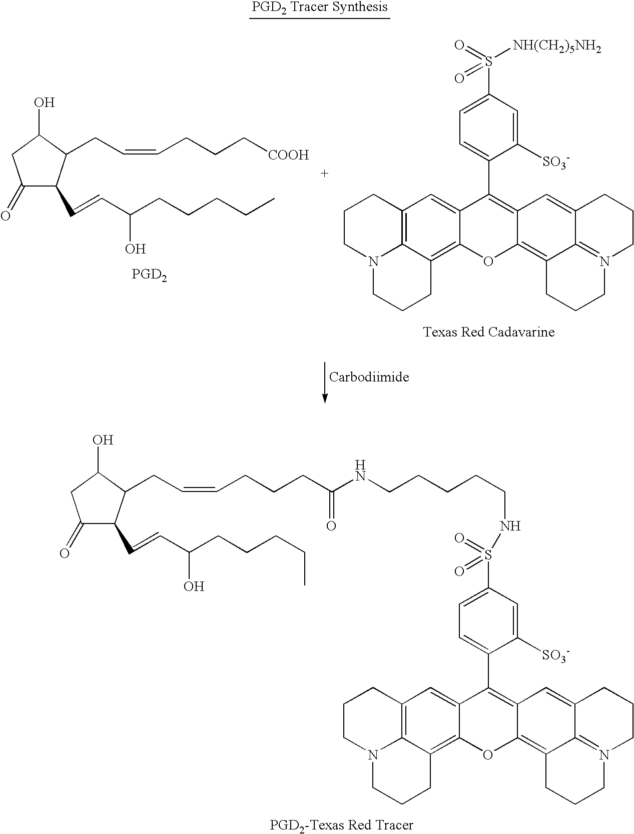

In this synthesis, a Texas Red Cadavarine (Molecular Probes) was added to a solution of the prostaglandin D2 (Cayman Chemicals) in dry methylene chloride. Dicyclohexylcarbodiimide (Sigma-Aldrich) was added and the reaction was stirred under nitrogen in the dark for 24 hours. Purification was performed by reverse phase HPLC chromatography using a water/acetonitrile gradient with 0.05% TFA as a modifier. Naturally, the linker used can be varied.

Method

This method is the same as that used in Example I, except that the enzyme utilized is hPGDS, and the prostaglandin product is PGD₂. Thus, as explained above, the enzyme and GSH were initially diluted together in reaction buffer containing K₂HPO₄ and KH₂PO₄ to make an enzyme solution. The compound or agent to be assayed was then placed in this phosphate buffered enzyme solution. Optionally, this solution can then be incubated. The duration of this incubation can vary from a few minutes up to over an hour. In this particular example, the enzyme solution was incubated for about 30 minutes.

The substrate PGH₂ in acetone was placed in a separate container at 4° C. The enzyme solution was then added to the container containing PGH₂ in order to start the reaction. This mixture was incubated for 30 seconds. Stop solution comprising FeCl₂ and citric acid was then mixed into the mixture to prevent spontaneous conversion of any remaining PGH₂ into PGE₂ or PGD₂. Detection Solution comprising an antibody having PGD₂ as an immunogen and Texas Red labeled PGD₂ (the tracer), was then added to the mixture, and the entire mixture was incubated. In this example, the mixture was incubated for about 120 minutes. However, the time period for this incubation as well as all other incubation periods described in this Example can be varied, depending on reagent concentrations.

A control mixture was prepared that was identical to the mixture containing the enzyme, wherein the control mixture was treated in a manner identical the mixture containing the enzyme, i.e., same incubation durations, etc. However, the control mixture did not contain the compound or agent being evaluated. The entire mixture and the control mixture were then illuminated with plane polarized light at a wavelength of 580 nm (the wavelength at which Texas Red is excited), and the fluorescence polarization of the mixture and the control mixture were measured using a fluorescence filter set with an excitation wavelength of 580 nm, and an emission wavelength of 620 nm. The fluorescence polarization measurement was made while the measuring instrument is in FP mode. Naturally, the wavelengths used will be dependent upon the fluorescence label used in the method. These two FP measurements were then compared to determine whether the fluorescence polarization measurement of the mixture containing the compound or agent is greater than the fluorescence measurement of the control solution. A fluorescence measurement of the mixture that is greater than the fluorescence polarization measurement of the control mixture indicates the compound or agent decreases the activity of hPGDS.

Results

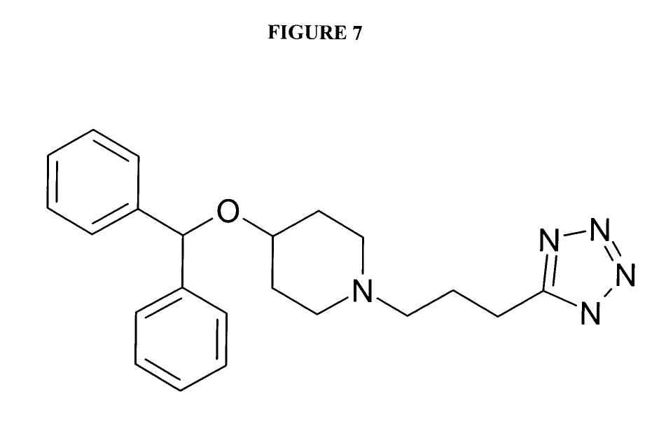

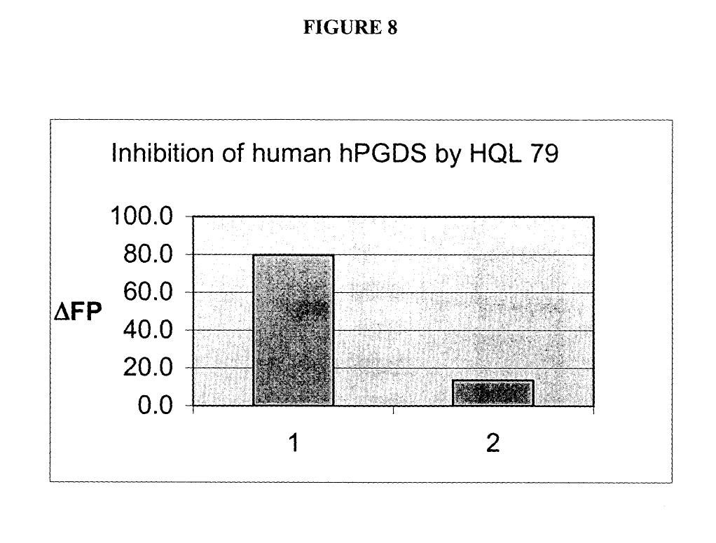

The assay described above was validated using HQL 79, a known inhibitor of hPGDS. HQL 79 is described in WO 95/01350. The structure of HQL 79 is set forth in FIG. 7. The results of this experiment are set forth in FIG. 8. These results clearly show that a method of the present invention detected that HQL 79 decreases the activity of hPGDS.

Conclusion

Examples I and II readily demonstrate that a method of the present invention is simple, does not require multiple washings or radioactive isotopes, and can readily be used in a high throughput manner, to determine whether a compound or agent decreases or inhibits the activity of a prostaglandin synthase, and in particular of hPGDS or mPGES.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A method for determining whether a compound or agent decreases the activity of a prostaglandin synthase selected from the group consisting of a microsomal prostaglandin E synthase (MPGES) and a hematopoietic prostaglandin D synthase (hPGDS) to react with a substrate to form a prostaglandin product, comprising the steps of:
(a) mixing the prostaglandin synthase with the substrate, a cofactor and the compound or agent;
(b) incubating the mixture of step (a) with a stop solution comprising an agent that prevents the spontaneous conversion of the substrate into the prostaglandin product;
(c) incubating the mixture of step (b) with a detection reagent comprising the prostaglandin product labeled with a fluorescence label, and an antibody having the prostaglandin product as an immunogen;
(d) illuminating the mixture of step (c) and a control mixture with plane polarized light having a wavelength at which the fluorescence label can be excited, and measuring the fluorescence polarization of the mixture of step (c) and the control mixture; and
(e) comparing the measurements of step (d),
wherein finding the fluorescence polarization measurement of the mixture (c) is greater than the fluorescence polarization measurement of control mixture indicates the compound or agent decreases the activity of the prostaglandin synthase.

2. The method of claim 1, wherein the prostaglandin synthase is microsomal prostaglandin E synthase (mPGES), the substrate is prostaglandin H₂ (PGH₂), the cofactor is glutathione (GSH), and the prostaglandin product is prostaglandin E₂ (PGE₂).

3. The method of claim 2, wherein the microsomal prostaglandin E synthase is human mPGES comprising the amino acid sequence of SEQ ID NO:2.

4. The method of claim 1, wherein the prostaglandin synthase is hematopoietic prostaglandin D synthase (hPGDS), the substrate is PGH₂, the cofactor is glutathione (GSH), and the prostaglandin product is prostaglandin D₂ (PGD₂).

5. The method of claim 4, wherein the hematopoietic prostaglandin D synthase is human hematopoietic prostaglandin D synthase and comprises the amino acid sequence of SEQ ID NO:4.

6. The method of claim 1, wherein the agent of the stop solution is FeCl₂.

7. The method of claim 1, wherein the fluorescence label comprises fluorescein, phycoerythrin (PE), Texas red (TR), rhodamine, a free lanthanide series salt, a chelated lanthanide series salt, BODIPY, ALEXA, or CyDye.

8. The method of claim 7, wherein the fluorescence label is Texas red (TR).

9. The method of claim 2, wherein the agent of the stop solution is FeCl₂.

10. The method of claim 9, wherein incubating step (b) has a duration of at least 30 seconds, and the incubating step (c) has a duration of at least 3 minutes.

11. The method of claim 10, wherein the fluorescence label comprises fluorescein, phycoerythrin (PE), Texas red (TR), rhodamine, a free lanthanide series salt, a chelated lanthanide series salt, BODIPY, ALEXA, or CyDye.

12. The method of claim 11, wherein the fluorescence label is Texas red (TR), and the wavelength of the plane polarized excitation light is 580±20 nm.

13. The method of claim 4, wherein the agent of the stop solution is FeCl₂.

14. The method of claim 13, wherein the fluorescence label comprises fluorescein, phycoerythrin (PE), Texas red (TR), rhodamine, a free lanthanide series salt, a chelated lanthanide series salt, BODIPY, ALEXA or CyDye.

15. The method of claim 14, wherein the fluorescence label is Texas red (TR), and the wavelength of the plane polarized excitation light is 580±20 nm.

16. A method for determining whether a compound or agent decreases the activity of a prostaglandin synthase selected from the group consisting of hematopoietic prostaglandin D synthase (hPGDS) and microsomal prostaglandin E synthase (mPGES) to react with a substrate to form a prostaglandin product, comprising the steps of:
(a) mixing the prostaglandin synthase with the substrate, a cofactor and the compound or agent;
(b) incubating the mixture of step (a) with a stop solution comprising an agent that prevents spontaneous conversion of unreacted substrate into prostaglandin product;
(c) incubating the mixture of step (b) with a detection reagent comprising the prostaglandin product labeled with Texas Red, and an antibody having the prostaglandin product as an immunogen;
(d) illuminating the mixture of step (c) and a control mixture with plane polarized light at a wavelength of 580±20 nm and measuring the fluorescence polarization of the mixture of step (c) and the control mixture at 620±20 nm; and
(e) comparing the measurements of step (d),
wherein finding the fluorescence polarization measurement of the mixture of step (c) is greater than the fluorescence polarization measurement of control mixture indicates the compound or agent decreases the activity of the prostaglandin synthase.

17. The method of claim 16, wherein the prostaglandin synthase is human microsomal prostaglandin E synthase (mPGES) comprising the amino acid sequence of SEQ ID NO:2, the substrate is prostaglandin H2 (PGH2), the cofactor is glutathione, the prostaglandin product is prostaglandin E2 (PGE2).

18. The method of claim 17, wherein the agent of the stop solution is FeCl2.

19. The method of claim 18, wherein the prostaglandin product labeled with Texas Red comprises a linker molecule to which the prostaglandin product and the Texas Red are bound.

20. The method of claim 19, wherein the linker molecule is selected from the group consisting of aminobutyric acid, aminocaproic acid, 7-aminoheptanoic acid, 8-aminocaprylic acid, Fmoc-aminocaproic acid, one or more β-alanines, an isothiocyanate group, a succinimidyl ester, a fulfonal halide, and a carboduimide.

21. The method of claim 20, wherein the linker molecule is a carboduimide.

22. The method of claim 16, wherein the prostaglandin synthase is human hematopoietic prostaglandin D synthase (hPGDS) comprising the amino acid sequence of SEQ ID NO:4, the substrate is PGH₂, the cofactor is glutathione, and the prostaglandin product is prostaglandin D2 (PGD2).

23. The method of claim 22, wherein the agent of the stop solution is FeCl2.

24. The method of claim 23, wherein the prostaglandin product labeled with Texas Red comprises a linker molecule to which the prostaglandin product and the Texas Red are bound.

25. The method of claim 24, wherein the linker molecule is selected from the group consisting of aminobutyric acid, aminocaproic acid, 7-aminoheptanoic acid, 8-aminocaprylic acid, Fmoc-aminocaproic acid, one or more β-alanines, an isothiocyante group, a succinimidyl ester, a sulfonal halide, and a carboduimide.

26. The method of claim 25, wherein the linker molecule is a carboduimide.

27. A method for determining whether a compound or agent decreases the activity of human microsomal prostaglandin E synthase (mPGES) comprising the amino acid sequence of SEQ ID NO:2 to react with its prostaglandin H2 (PGH₂) substrate to form prostaglandin E₂ (PGE₂), the method comprising the steps of:
(a) mixing the mPGES with PGH₂, glutathione, and the compound or agent;
(b) incubating the mixture of step (a) with a stop solution comprising FeCl₂;
(c) incubating the mixture of step (b) with a detection reagent comprising PGE₂ labeled with Texas Red, and an antibody having PGE₂ as an immunogen;
(d) illuminating the mixture of step (c) and a control mixture with plane polarized light at a wavelength of 580±20 nm and measuring the fluorescence polarization of the mixture of step (c) and the control mixture; and
(e) comparing the measurements of step (d),
wherein finding the fluorescence polarization measurement of the mixture of step (c) is greater than the fluorescence polarization measurement of the control mixture indicates the compound or agent decreases the activity of the mPGES.

28. A method for determining whether a compound or agent decreases the activity of human hematopoietic prostaglandin D synthase (hPGDS) comprising the amino acid sequence of SEQ ID NO:4 to react with its prostaglandin H₂ (PGH₂) substrate to form prostaglandin D₂ (PGD₂), comprising the steps of:
(a) mixing hPGDS with PGH₂, glutathione and the compound or agent;
(b) incubating the mixture of step (a) with a stop solution comprising FeCl₂;
(c) incubating the mixture of step (b) with a detection reagent comprising PGD₂ labeled with Texas Red, and an antibody having PGD₂ as an immunogen;
(d) illuminating the mixture of step (c) and a control mixture with plane polarized light at a wavelength of 580±20 nm and measuring the fluorescence polarization of the mixture of step (c) and the control mixture; and
(e) comparing the measurements of step (d),
wherein finding the fluorescence polarization measurement of the mixture of step (c) is greater than the fluorescence polarization measurement of control mixture indicates the compound or agent decreases the activity of hPGDS.


SEQUENCE LISTING (4 sequences)

SEQ ID NO: 1 - DNA, 459 bp, Homo sapiens
atgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagcgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtga

SEQ ID NO: 2 - protein, 152 aa, Homo sapiens
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 3 - DNA, 600 bp, Homo sapiens
atgccaaactacaaactcacttattttaatatgagggggagagcagaaattattcgttacatatttgcttatttggacatacagtatgaagaccacagaatagaacaagctgactggcctgaaatcaaatcaactctcccatttggaaaaatccccattttggaagttgatggacttactcttcaccagagcctagcaatagcaagatatttgaccaaaaacacagatttggctggaaacacagaaatggaacaatgtcatgttgatgctattgtggacactctggatgatttcatgtcatgttttccttgggcagagaaaaagcaagatgtgaaagagcagatgttcaatgagctgctcacgtataatgcgcctcatcttatgcaagacttggacacatatttaggggggagagaatggcttattggtaactctgtaacttgggcagacttctactgggagatttgcagtaccacacttttggtctttaagcctgacctgttagacaaccatccaaggctggtgactttacggaagaaagtccaagccattcctgccgtcgctaactggataaaacgaaggccccaaaccaaactctag

SEQ ID NO: 4 - protein, 199 aa, Homo sapiens
MPNYKLTYFNMRGRAEIIRYIFAYLDIQYEDHRIEQADWPEIKSTLPFGKIPILEVDGLTLHQSLAIARYLTKNTDLAGNTEMEQCHVDAIVDTLDDFMSCFPWAEKKQDVKEQMFNELLTYNAPHLMQDLDTYLGGREWLIGNSVTWADFYWEICSTTLLVFKPDLLDNHPRLVTLRKKVQAIPAVANWIKRRPQTKL